(19) United States
(12) Patent Application Publication
(10) Pub. No.: US 2017/0258308 A1
(43) Pub. Date: Sep. 14, 2017

(54) LIGHTING ASSEMBLY FOR CAMERA MODULE OF AN ENDOSCOPE

(71) Applicant: CHIYI TECHNOLOGY CO., LTD., NEW TAIPEI CITY (TW)

(72) Inventor: YEN TSUNG LIN, NEW TAIPEI CITY (TW)

(21) Appl. No.: 15/332,019

(22) Filed: Oct. 24, 2016

(30) Foreign Application Priority Data
Mar. 11, 2016 (TW) 105203353

(51) Int. Cl. A61B 1/06; A61B 1/05; A61B 1/00; G02B 23/24; H04N 5/225

(52) U.S. Cl. CPC A61B 1/0607; G02B 23/2461; H04N 5/2256; H04N 5/2257; A61B 1/00066; A61B 1/00105; A61B 1/05; A61B 1/0676; A61B 1/0684; A61B 1/0011; G02B 23/2484; H04N 2005/2255

(57) ABSTRACT

The present invention relates to an improved lighting assembly for camera module of an endoscope. The camera module is received in a cylinder body, which is connected with the tail end of a tube of the endoscope. The cylinder body is provided with two side openings covered by related transparent caps. Several lighting sources are mounted in the cylinder body at the position near the side openings that emits side lightings to increase whole lighting effect in the working cavity and facilitates to obtain clear image captured from camera module. Moreover, the front cover is provided with an annular misty portion related to the position above the front lighting sources around the camera module. Hence, the forward emitting lighting will be foggy that can thus avoid from the reflected lighting spots and benefit the use of the present endoscope to catch clear images of patient's inner cavity.

### BACKGROUND OF THE INVENTION

The endoscope normally utilized on medical surgery procedures that is harmless to people, includes a control handle with a long tube for inserting into human's cavity. The tail portion of the tube is provided with a camera module and a lighting source therein to catch image data of organs in human's body. A prior lighting source is used of a glass-fiber to transport the light made from a lighting source mounted in the control handle. Since the glass-fiber is possibly broken, it is troublesome when producing, and is more harmful and dangerous if the glass-fiber is broken during proceeding a medical surgery. In late years, it had developed a new lighting structure for the endoscope. As shown in FIG. 1, this known structure includes a cylinder body (1), which has provided with several lighting sources (11), such as LEDS, at front face around the camera module (12). In use, the light of the lighting of the sources (11) is directly projecting forward and will be reflected because the cavity of human has glossy surface and is full of liquid that will occur a lighting spot on the signal of the image captured by the camera. It leads a bland area and increases risk of surgery. Moreover, since all lighting sources are mounted at the front portion of the cylinder body, the produced high heat may cause another danger that hurts the patient. Hence, the known designs are not utilized and should be improved.

### SUMMARY OF THE INVENTION

The present invention is aimed to design an improved lighting assembly for camera module of an endoscope which can overcome the drawbacks mentioned in prior art. The camera module is received in a cylinder body, which is connected with the tail end of the tube of the endoscope. The cylinder body has provided with two side openings covered by related transparent caps. Several lighting sources are mounted in the cylinder body related to the position of the openings that provides side lights to increase whole lighting effect in the working cavity and will facilitate to obtain clear image captured from camera module. Moreover, the front cover is provided an annular misty portion related to the position above the front lighting sources. The forward emitting light will be foggy that can thus avoid from the reflected lighting spots to overcome the drawback of the convention endoscope. Now, accompanying with drawings, the character and structure of the present invention will be disclosed as following.

### BRIEF DESCRIPTION OF THE DRAWINGS

FIG. 1 is a perspective view showing the lighting assembly, the camera module, and the cylinder body of a conventional endoscope.

FIG. 2 is a perspective view showing the improved lighting assembly, the camera module, and the cylinder body according to the present invention.

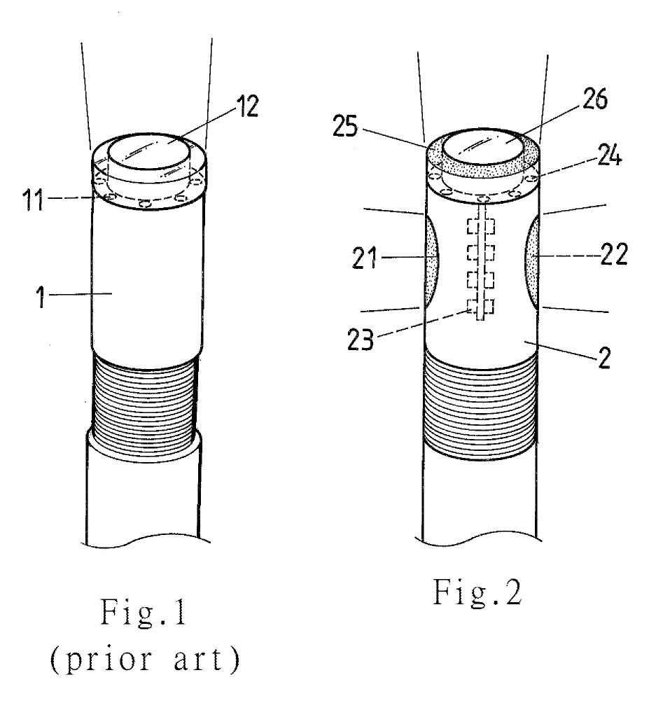

### DETAILED DESCRIPTION OF THE PREFERRED EMBODIMENT

Referring to FIG. 2, the present invention relates to an improved lighting assembly for camera module of an endoscope. The camera module (26) is received in the cylinder body (2) at the tail end of the endoscope. The character of the invention is to provide the cylinder body (2) with two side openings (21), which are covered with a related transparent cap (22). In cylinder body (2), there are several lighting sources (23), such as LEDS, provided respectively at the position near the openings (21). The front cover of the cylinder body (2) is further provided with an annular misty portion (25), which is at the position above the front annular located lighting sources (24). The annular misty portion (25) can be made by coating foggy glue on the front cover of the cylinder body (2)

In application, it is to be understood that the side lighting sources (23) can emit light through the transparent caps (22) outwards that provides additional lighting in the cavity for benefiting the camera module (26) to catch clearer images. Moreover, the front light from the front lighting sources (24) will be foggy to avoid from the reflecting light spots during taking inner image of human's cavity. Hence, the medical surgery will be more safe and efficient. In further embodiment, it is possible to design a control system to adjust the strength of the lighting sources of the present invention to prevent high heat occurred from gathering lighting sources.

Above all, the present obtains utility in no doubt. It is obviously utilized and effective for patient during medical procedures. Thus, it is to be understood that the present is allowable.

What is claimed is:

1. An improved lighting assembly for camera module of an endoscope, which including a control handle having a tube connecting the tail end with a cylinder body receiving inside camera module, and the characteristic is in that:
the cylinder body being provided with two side openings covered with related transparent caps, several side lighting sources being mounted in the cylinder body near the side openings, and
the cylinder body being provided with an annular misty portion at front cap, which is at the position above the front lighting sources annular located around the camera module.

2. The improved lighting assembly for camera module of an endoscope as claimed in claim 1, wherein the lighting source is LED.

3. The improved lighting assembly for camera module of an endoscope as claimed in claim 1, wherein the annular misty portion is completed by coating foggy glue on the front cover of the cylinder body.
